(19) United States
(12) Patent Application Publication
(10) Pub. No.: US 2019/0166774 A1
(43) Pub. Date: Jun. 6, 2019

(54) METHOD TO INCREASE CROP PLANT FOLIAGE PRODUCTIVITY

(71) Applicant: The Regents of the University of California, Oakland, CA (US)

(72) Inventors: Anastasios Melis, El Cerrito, CA (US); Henning Kirst, Berkeley, CA (US); Peggy G. Lemaux, Moraga, CA (US); Stephane T. Gabilly, Walnut Creek, CA (US)

(73) Assignee: The Regents of the University of California, Oakland, CA (US)

(21) Appl. No.: 16/313,864

(22) PCT Filed: Jun. 28, 2017
(86) PCT No.: PCT/US2017/039685

§ 371 Date: Dec. 27, 2018

Related U.S. Application Data
(60) Provisional application No. 62/356,185, filed on Jun. 29, 2016.

(51) Int. Cl. A01G 22/00; A01H 5/12; A01H 6/82; A01G 17/00; A01G 22/45; A01G 22/20; A01G 22/25; A01G 22/05; A01G 22/50; A01G 17/02; A01G 22/40

(52) U.S. Cl. CPC A01G 22/00; A01H 5/12; A01H 6/823; A01G 17/005; A01G 22/45; A01G 22/40; A01G 22/25; A01G 22/05; A01G 22/50; A01G 17/02; A01G 22/20

(57) ABSTRACT

Methods and compositions are disclosed to improve biomass accumulation in Truncated Light-harvesting Antenna (TLA) crop plant canopies as compared to biomass accumulation measured in wild-type counterparts grown under the same high canopy-density, agronomic, and ambient sunlight conditions.

### CROSS-REFERENCE TO RELATED APPLICATIONS

This application claims benefit of priority to U.S. Provisional Application No. 62/356,185, filed Jun. 29, 2016, which is herein incorporated by reference for all purposes.

### STATEMENT AS TO RIGHT TO INVENTIONS MADE UNDER FEDERALLY SPONSORED RESEARCH AND DEVELOPMENT

This invention was made with government support under Contract No. DE-AR0000204 awarded by the U.S. Department of Energy. The government has certain rights in this invention.

### BACKGROUND OF THE INVENTION

Photosynthetic organisms, including bacteria, algae, and plants, have evolved extensive arrays of light-harvesting pigments, comprising chlorophylls, carotenoids, and bilins that absorb sunlight and transfer the excitation energy to photochemical reaction centers. The latter convert the absorbed irradiance to chemical energy via the photochemical charge separation reaction, which is viewed as the beginning step of photosynthesis. The evolution of sizable arrays of light-harvesting antennae in all photosynthetic systems confers a selective advantage for the organism in nature, where sunlight intensity is often the growth-limiting parameter. Successful competition in nature requires capturing more sunlight for self, even if wasted, and preventing light capture by competing neighbors (Melis 2009). Consequently, top layers of plant canopies and upper layers of microalgae in high-density monocultures absorb sunlight far in excess of what is needed to saturate photosynthesis (Melis et al. 1999; Polle et al. 2003; Melis 2009; Ort et al. 2011), Excess absorbed irradiance is dissipated in an orderly manner by the photosystems via non-photochemical quenching (NPQ) mechanisms, which evolved to protect the photosynthetic apparatus and prevent photosensitized bleaching (Müller et al. 2001; Ruban 2016).

In organisms of oxygenic photosynthesis, large arrays of light-harvesting pigment-protein complexes are peripheral components of photosystem-1 (PSI) and photosystem-II (PSII). Minimizing, or truncating, the chlorophyll antenna size of the photosystems can improve photosynthetic solar energy conversion efficiency and productivity in high foliage density monocultures (reviewed in Melis 2009). The rationale is that individual chloroplasts and photosystems with a smaller chlorophyll antenna size in the upper canopy leaves will have a diminished probability of absorbing sunlight, thereby permitting greater penetration and a more uniform distribution and utilization of irradiance throughout the foliage of the monoculture. Such altered optical properties alleviate over-absorption and wasteful dissipation of sunlight by the upper canopy and enhance photosynthetic productivity of the foliage of the monoculture as a whole. The Truncated Light-harvesting Antenna (TLA) concept, referring to a smaller than wild type chlorophyll antenna size of the photosystems, has found application and noteworthy success in the case of high-density cultivation of microalgae (Nakajima and Ueda 1997, 1999; Melis et al. 1999; Nakajima and Itayama 2003; Polle et al. 2003; Mussgnug et al. 2007) and cyanobacteria (Kirst et al. 2014), but has not been applied to plant canopies.

### BRIEF SUMMARY OF THE INVENTION

In one aspect, this disclosure relates to high-density plant canopies and is based, in part on the discovery that greater biomass accumulation occurs in TLA plant canopies over that measured in wild-type counterparts grown under the same agronomic and ambient conditions. Distinct plant anatomical appearance differences also occur.

### BRIEF DESCRIPTION OF THE DRAWINGS

FIG. 1 Visual appearance and coloration of Nicotiana tabacum wild type and TLA leaves. Wild type (WT) tobacco leaves have dark green coloration whereas the TLA tobacco leaves have light green coloration.

FIG. 2 Coomassie stain of total protein extracts from Nicotiana tabacum wild type (WT) and TLA leaves resolved by SDS-PAGE. Lanes were loaded with 2 μg chlorophyll (a and b) for the TLA analysis and with 0.5, 1.0, or 2.0 μg chlorophyll (a and b) for the wild type (WT) analysis. On a chlorophyll basis, the TLA sample contained more RBCL, the large subunit of RubisCO, than the wild type. However, the TLA sample contained substantially lower levels of LHC-II, apoproteins of the major light-harvesting complex of PSII, than the wild type.

FIG. 3 Western blot analysis of total protein extracts from Nicotiana tabacum wild type (WT) and TLA leaves. Total protein extracts were resolved by SDS-PAGE, transferred onto nitrocellulose membranes, and probed with specific polyclonal antibodies raised against the PsaA/PsaB photosystem-I reaction center proteins (PSI RC), the PsbD photosystem-II reaction center protein (D2), or the light-harvesting chlorophyll-proteins LHCB1 and LHCB2 of PSII. Note the approximately equal levels of D2 protein (relative measure of PSII) in the WT and TLA lanes, and the substantially lower PSI RC and LHCB protein levels in the TLA tobacco relative to the wild type.

FIG. 4 Light-saturation curves of photosynthesis in Nicotiana tabacum wild type (dark green) and TLA leaves (light green) at ambient CO₂ concentration (400 μmol mol⁻¹). Saturation of photosynthesis in the wild type was estimated to occur at 425 μmol photons m-2 s-1, whereas in the TLA mutant saturation occurred at 635 μmol photons m-2 s-1.

FIG. 5 Visual appearance of Nicotiana tabacum wild type and TLA canopies, shown near the end of their respective growth period. The wild type tobacco leaves have a dark green coloration (upper panel) and the TLA tobacco leaves have a light coloration (lower panel). The wild type plants also have longer internode distances with fewer leaves in their upper canopy of the wild type as compared with that of the TLA plants. The overall foliage density is higher in the TLA monoculture compared to the wild type.

FIGS. 6A and 6B (6A) Average values of biomass harvested from three different pairs of wild type and TLA monocultures grown at different periods of time during the growth season in the greenhouse. TLA monocultures produced about 25% more biomass than the corresponding wild-type monocultures. (6B) Biomass accumulation by individual wild type—TLA pairs of monocultures, showing variation among the absolute yields of the three separate pairs, depending on time of the year and greenhouse location of the monocultures, but in all cases the TLA monoculture produced more biomass than the corresponding wild type.

FIG. 7. Schematic of traditional plant (tobacco) agronomy shows the typical 18-inch distance between adjacent plants, translating into an average of 4.35 plants cultivated per m². This planting density prevents individual plants from unduly shading one-another.

FIG. 8. Schematic of modified tobacco agronomy shows a narrow 9-inch distance between adjacent plants, translating into a monoculture density of 14.9 plants per m². This substantially greater planting density takes advantage of the TLA property of the photosystems, and of the ensuing lower chlorophyll content per leaf area, properties that afford a greater transmittance of sunlight through the foliage, thereby enhancing monoculture productivity.

FIG. 9. Schematic of alternative plant (tobacco) agronomy shows planting in rows of three with an 18-inch distance between the groups of three. A 9-inch distance from each other separates individual plants in a group of three. This configuration translates into 12.7 plants per m². This planting density also benefits from the TLA size of the photosystems, and of the ensuing lower chlorophyll content per leaf area, properties that afford a greater transmittance of sunlight through the foliage, thereby enhancing monoculture productivity.

### DETAILED DESCRIPTION

### Terminology

In the context of this invention, a “Truncated Light-harvesting Antenna” or “TLA” plant has a genetic modification that results in a smaller antenna size of the photosystems compared to a wild-type counterpart plant of the same strain that does not have the genetic modification. In the present disclosure, TLA antennae are typically less than about 80%, or less than about 75%, less than about 70%, less than about 65%, less than about 60%, less than about 55%, or less than about 50% of the antenna size of the corresponding wild type plant, and are at least about 20%, or at least about 25% or about 30%, of the antenna size of the corresponding wild type plant.

A “broad” leaf in the context of this disclosure refers to a leaf having a length-to-width ratio of less than 5:1.

A “blade-like leaf” in the context of this disclosure refers to a leaving having a length-to-width ratio of 5:1 or greater.

The terms “foliage” as used herein refers to the totality of the leaves and other green tissues of a plant, shrub, or tree, including leaves on the stems or branches, which collectively contribute to photosynthesis and plant growth and development.

The term “canopy” and “upper canopy” as used herein refers to the upper layer of leaves in a monoculture that are directly exposed to the sun and may over-absorb and wastefully dissipate sunlight energy, while shading leaves that are lower or internal to the foliage. A plant canopy changes dynamically, as plants grow and develop.

As used herein “bright sunlight” refers to incident photosynthetically active radiation (PAR, or visible light) exceeding 500 μmol photons per m² per s¹.

As used herein, the term “about” means that a value may vary ±15%, ±10% or ±5% and remain within the scope of the invention.

A “plant” as used herein refers to a whole plant that is cultivated. The class of plants that can be used in the method of the disclosure includes monocots and dicots and any plant that is planted in soil for culture.

### TLA Plants

The present disclosure is based in part on the discovery that cultivating TLA plants so that they are closely spaced such that there is significant overlap of the leaves results in plants that have increased biomass compared to wild-type counterpart plants.

In the present disclosure, a TLA plant harbors a mutation that results in a decrease in antenna size relative to the wild-type plant that does not have such a mutation. A TLA plant has an antenna size that are decreased by at least 20%, at least 25%, typically at least 30%, at least 35%, at least 40%, at least 45%, at least 50%, or at least 60%, at least 70%, or 80% compared to the counterpart wild type plant. Antenna size of the photosystems can be measured using any method. For example, antenna size of the photosystems in situ can be determined using the spectrophotometric and kinetic method of Melis (see, e.g., Phil. Trans. R. Soc. Lond. B 323: 397-409, 1989.) Illustrative results of antenna size measurements are described in the examples.

TLA plants that have a mutation results in decreased antenna size are known. For example, mutations that result in small or truncated antenna size can in occur in genes such as TLA1 (U.S. Pat. No. 7,745,696), TLA2 (US Patent Application 20140295448), TLA3 (Kirst et al. 2012), CAO (Chlorophyllide a oxygenase; Ghirardi et al. 1986; Polle et al. 2000), Mg-chelatase subunit I, and CHL-I (Hansson et al. 1999; Fitzmaurice et al. 1999). In some embodiments, a TLA plant, i.e., a plant having reduced chlorophyll antenna size, has a mutation in a gene as indicated in the references cited in the preceding sentence where the mutated gene is unknown. In some embodiments, a TLA plant has a genetic modification resulting in decreased chlorophyll antenna size where the mutation has not been identified. Example of plant lines that having decreased antenna size are provided in Table 3. Methods of identifying or generating such plants are described in the references cited herein, each of which is herein incorporated by reference for this purpose.

TLA plants that are planted at high density in accordance with the disclosure, exhibit a chlorophyll a to chlorophyll b ratio that is increased from that of wild type plants, which wildtype ratio is typically about 3±1. Thus, in some embodiments, TLA plants cultured as described herein have a chlorophyll a to chlorophyll b of at least 4±1, 6±1, 8±1, 12±1, or 20±1. In some embodiments, the chlorophyll a to chlorophyll b ratio is greater than 20±1, e.g., 25±1, or greater. In some embodiments, TLA plants do not have measureable amounts of chlorophyll h. The chlorophyll a and chlorophyll b content and compositions of leaves can be determined using any method. Often, chlorophyll a and chlorophyll b content are measured upon pigment extraction from the leaves in organic solvent, typically methanol ethanol, or acetone, and quantified by their specific absorbance in the red region of the spectrum, as per the Arnon method (1949). Example using methanol extraction and quantification of the chlorophylls spectrophotometrically is provided in the Examples section of the present disclosure.

A “grouping” of plants in the context of the present disclosure refers to the overall pattern in which plants are planted. Each grouping contains individual plants. Often, the individual plants are spaced such that plants within the grouping are closer to one another than are the grouping to one another. For example, a grouping can be rows, clusters, circles, or other geometrical configurations.

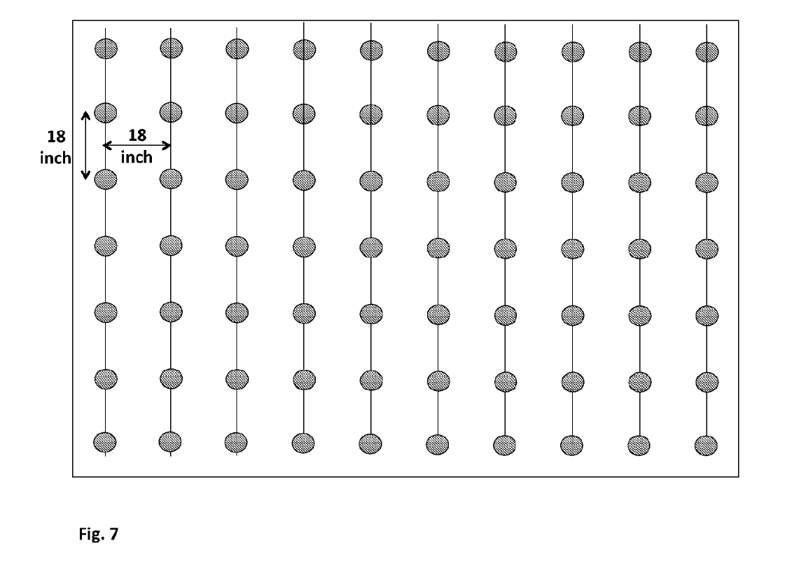

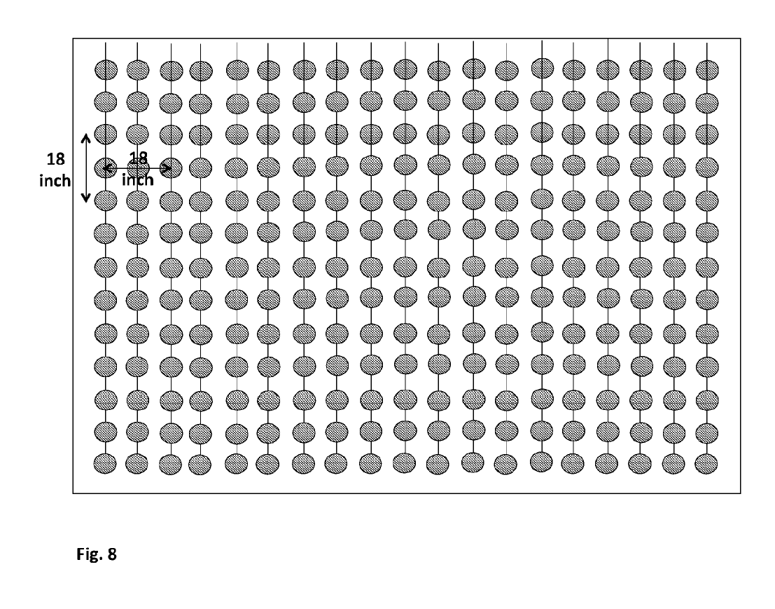

In typical agronomic practice, e.g., in planting wild type tobacco and grape vine, individual plants are arranged in rows, separated by about 18 inches from each other, with rows also separated by about 18 inches from one another (see, FIG. 7 by way of illustration). This illustrative agronomic configuration, e.g., for wild type tobacco and grape vine plants, results in a field with a plant density of about 4 to 4.5 plants per m². In order to obtain greater biomass per surface area compared to a wild-type plant, TLA plants, e.g., tobacco or grape vine plants. Plants may be planted in rows, circles, or other configurations. Typically, TLA plants such as tobacco or grape vine plants, within a grouping, such as a row or cluster, are separated by less than about 9 inches, whereas the groupings of plants are separated by less than about 18 inches and as close as 9 inches from one another, with the rows also separate by less than about 18 inches and as close as about 9 inches from each other (see, FIG. 8 by way of illustration). This agronomic configuration results in a field plant density of about 15 plants per m². TLA plants are capable of making efficient sunlight utilization under such high planting density, thereby supporting growth and productivity that exceeds the wild type under the same high-density monoculture agronomic conditions. Whereas the schematic of FIG. 8 depicts a better than 300% increase in planting density, alternative groupings may result in planting density increase by 50%, 100%, or by 200% over what could be practiced with their wild type counterpart plants.

The description in the preceding paragraph is described using tobacco and grape vine plants as illustrative embodiments; however, plant density increases by 100%, or 200%, or 300% can be achieved with other TLA crop species having reduced chlorophyll antenna size (see, e.g., Table 3) or can be genetically engineered using such techniques as random mutagenesis coupled with selection for TLA antenna size as described herein or by specifically targeting a gene known to influence TLA antenna size, e.g., TLA1, TLA2, TLA3, CAO, Mg-chelatase subunit I, and CHL-I.

TLA plants cultivated in accordance with this disclosure, typically generate a. harvestable total biomass that is at least about 15%, at least about 20%, at least about 25%, at least about 50%, or at least about 100%, or greater, than a counterpart wild type plant monoculture grown under the same high-density monoculture cultivation conditions.

In certain embodiments of the invention, the plant is corn, switchgrass, sorghum, miscanthus, sugarcane, alfalfa, wheat, soy, cotton, barley, turf grass, tobacco, potato, bamboo, rape, sugar beet, sunflower, millet, bean, tomato, canola, or grape vine.

### EXAMPLES

### Materials and Methods

### Plant Material

Nicotiana tabacum, cv John William's Broadleaf, and the yellow-green mutant Su/su (Homann and Schmid 1967; Okabe et al. 1977) were grown in the greenhouse under ambient sunlight conditions. Seeds were kindly provided by Dr. Georg H. Schmid, University of Bielefeld, Germany. Prior analysis has shown the yellow-green Su/su mutant to have a substantially smaller than wild type light-harvesting antenna size (Melis and Thielen 1980; Thielen and van Gorkom 1981). Hence, the yellow-green mutant Su/su mutant will be referred to as Truncated Light-harvesting Antenna tobacco (TLA tobacco).

### Pigment Determination and Leaf Protein Analysis

The chlorophyll and carotenoid concentration of leaves was determined spectrophotometrically in 100% methanol extracts of tobacco leaves according to Lichtenthaler (1987). Protein analyses were conducted with leaf extracts resolved in precast SDS-PAGE gels (BIO-RAD). Loading of samples was based on chlorophyll content.

### Chloroplast and Thylakoid Membrane Isolation

Leaves were homogenized in ice-cold chloroplast isolation buffer containing 0.4 M sucrose, 50 mM Tricine (pH 7.8), 10 mM NaCl, 5 mM MgCl₂, 0.2% polyvinylpyrrolidone 40, 1% sodium ascorbate, 1 mM aminocaproic acid, 1 mM aminobenzamidine and 100 μM phenylmethylsulfonyl fluoride (PMSF). The suspension was filtered to separate unbroken leaf pieces from the cell lysate. Chloroplasts were pelleted by centrifugation at 5,000 g for 10 min and washed twice with chilled chloroplast isolation buffer. For thylakoid membrane isolation, chloroplasts were lysed by re-suspension in a glass homogenizer in hypotonic buffer containing 50 mM Tricine (pH 7.8), 10 mM NaCl, 5 mM MgCl₂, 0.2% polyvinylpyrrolidone 40, 1% sodium ascorbate, 1 mM aminocaproic acid, 1 mM aminobenzamidine and 100 μM phenylmethylsulfonyl fluoride. Thylakoid membranes were then pelleted by centrifugation at 75,000 g for 45 min at 4° C. Membranes were resuspended in 50 mM Tricine (pH 7.8), 10 mM NaCl, 5 mM MgCl₂ for spectrophotometric analysis.

### Spectrophotometric and Kinetic Analyses

The light-minus-dark absorbance difference signal at 700 nm (P700) for PSI, and 320 nm (QA) for PSII (Melis and Brown 1980; Melis 1989) were measured with a laboratory constructed sensitive absorbance difference spectrophotometer. Photosystems quantification estimates were then based on the measurements of P700 and QA. The functional light-harvesting chlorophyll antenna size of PSI and PSII was estimated from the rate constant of P700 photo-oxidation and QA photoreduction, measured upon weak green actinic illumination of isolated and DCMU-poisoned thylakoid membranes (Melis 1989).

### Measurement of Photosynthesis

Photosynthetic gas exchange measurements were made using a portable open gas analysis device (LI-6400, Li-Cor Inc., Lincoln, Nebr., USA). Light response curves were measured on the youngest and second youngest fully expanded and attached leaves of at least 2 different plants from the same canopy. Leaf temperature and CO₂ concentration in the leaf chamber were 25° C. and 400 μmol mol⁻¹ (ambient CO₂ concentration), respectively. The vapor pressure deficit was maintained below 1 kPa. The adaxial side of the leaf was illuminated by the light source (10% blue, 90% red). The starting light intensity was 1500 μmol photons m⁻² s⁻¹, then lowered to 1200, 1000, 800, 600, 400, 200, 100, 50, and 0. Measurements at various light intensities were recorded after the rate of photosynthesis reached steady state (after about 10 minutes).

### Foliage Density Biomass Accumulation Experiments

Heterozygous (Su/su) Nicotiana tabacum seeds were sprinkled on soil in seed pots in the greenhouse nursery for germination. Wild type (dark green), TLA (light-green), and white/lethal phenotypes emanated from the heterozygous seeds. Viable seedlings were transferred to 4×4 inch peat pots (soil course: Sunshine Mix #1, Sun Gro Horticulture, McClellan Park, Calif. 95652) for primary growth as individual plants (2-to-3 weeks). They were then transferred into soil in 2-gallon pots having a 9-inch diameter. Twenty-five (25) such wild type or TLA pots were placed adjacent to each other in a 5×5 configuration for growth and biomass production measurements, comprising a 1.3 m² (2.025 inch) plant monoculture. Such high-density monocultures with plants separated from one another by 9-inches were allowed to develop until about half of the plants showed signs of bolting. The biomass of the entire monoculture (all 25 plants) was then harvested and the fresh weight was recorded. Dry biomass weight was estimated by assuming a 12% wet-to-dry conversion factor. Experiments were conducted in pairs, with a wild type and TLA monoculture growing during the same period of time, in the same greenhouse and grown under identical ambient conditions.

### Results

### Pigmentation Characteristics of WT and the TLA Tobacco

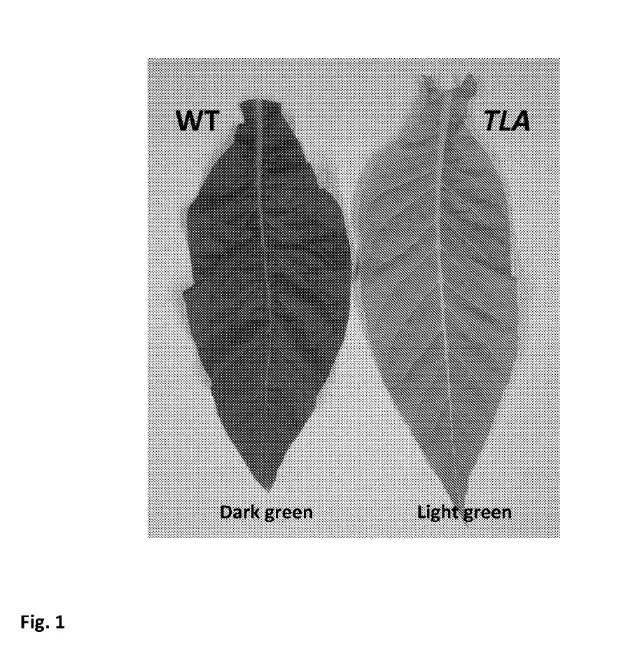

Leaves of the TLA tobacco displayed a distinct light-green coloration compared to the dark green wild type strain (FIG. 1). Analysis of the pigment content showed significantly lower total chlorophyll per leaf area in the TLA leaves compared to the wild type. The total chlorophyll (Chl) content of wild type leaves was about 24.9 mg cm⁻², whereas the TLA strain contained only about 12.1 mg cm⁻² (Table 1). The Chl b content of the TLA leaves was disproportionately lower relative to the lowering in Chl a. Measurements showed that Chl a content in the TLA leaves was lowered to 57% of the wild type, whereas Chl h content was lowered to about 23% of wild type. In consequence, the Chl a/Chl b ratio of the TLA was elevated to a ratio of 8:1, relative to the wild type that displayed a Chl a/Chl b ratio of about 3:1. The latter is typical for the fully developed photosynthetic apparatus in the leaves of green plants (Anderson 1986), whereas a substantially greater Chl a/Chl b ratio of the TLA leaves indicates a truncated light-harvesting Chl antenna size. The carotenoid content also differed between wild type and TLA tobacco. Total carotenoid content in the TLA strain was slightly lower, about 85% of that in the wild type (Table 1), resulting in a Car/Chl ratio of 0.16 in the wild type, but 0.29 in the TLA mutant (Table 1). These results are consistent with the notion of a truncated light-harvesting Chl antenna size in the tobacco mutant.

### Photochemical Apparatus Organization Wild Type and TLA Tobacco

The concentration of the photosystems in isolated tobacco thylakoid membranes was measured using the sensitive absorbance difference spectrophotometric method (Melis and Brown 1980) from the amplitude of the light minus dark absorbance difference signal at 700 nm (P700) for PSI, and 320 nm (QA) for PSII. Isolated thylakoid membranes from the wild-type tobacco showed an overall Chl/QA ratio of 383:1, whereas this ratio dropped to 129:1 for the TLA mutant (Table 2). Moreover, the overall Chl/700 ratio was 412:1. for the wild type, whereas this ratio dropped to 378:1 for the TLA mutant. These quantifications translated into a photosystem (PSII:PSI) molar ratio of 1.08:1 for the wild type and 2.93:1 for the TLA mutant. Enhancement of the PSII:PSI molar ratio in the TLA tobacco is a compensation response of the chloroplasts to the disproportionately smaller antenna size of PSII, resulting from the disproportionate lowering of Chl b over that for Chl a in these mutants (Greene et al. 1988).

### Functional Light-Harvesting Chl Antenna Size of the Photosystems

Chlorophyll b pigments are exclusively present in the light-harvesting antenna proteins but not in the core photosystem or reaction center complexes. The higher Chl a/Chl b ratio in the TLA strain compared to the wild type suggested a lower amount of Chl a-b light harvesting antenna proteins in the TLA chloroplasts. The functional light-harvesting Chl antenna size of PSI and PSII was measured from the kinetics of P700 photo-oxidation and. QA photoreduction kinetics, respectively (Melis 1989). Results from these measurements are summarized in Table 2. in the wild type, due to the biphasic kinetics of QA photoreduction, two rates constants of PSII photochemistry were discerned, K_{II} α[=8.4 s⁻¹] and K_{II} β[=5.1 reflecting the well-known PSII heterogeneity of PSII-α and PSII-β centers in chloroplasts (Melis and Homann 1976). Under the same weak actinic illumination conditions, the rate constant K₁ of PSI photochemistry was measured to be equal to 7.2 s⁻¹. These rate constants are a measure of the rate of light absorption by the photosystems, and are directly proportional to the specific photosystem functional light-harvesting Chl (a+b) antenna size. The precise number of the functional PSII chlorophylls was derived (Melis 1989) as 242 Chl for PSII-α and 148 Chl for PSII-β. The average functional PSII chlorophyll antenna size in the wild type was estimated to be 191 molecules. Similarly, the precise number of the functional PSI chlorophylls was estimated to be 207 in the wild type (Table 2). In the TLA mutant, all photosystem light-harvesting Chl (a+b) antenna sizes were smaller with 119 Chl for PSII-α and 86 Chl for PSII-β (average PSII Chl antenna size of 93 molecules) and a PSI average functional Chl antenna size of 104 molecules (Table 2). These results clearly show the effect of Chl deficiency on the functional antenna size of the photosystems, and are consistent with measurements conducted earlier (Melis and Thielen 1980; Thielen and van Gorkom 1981).

### Protein Analysis

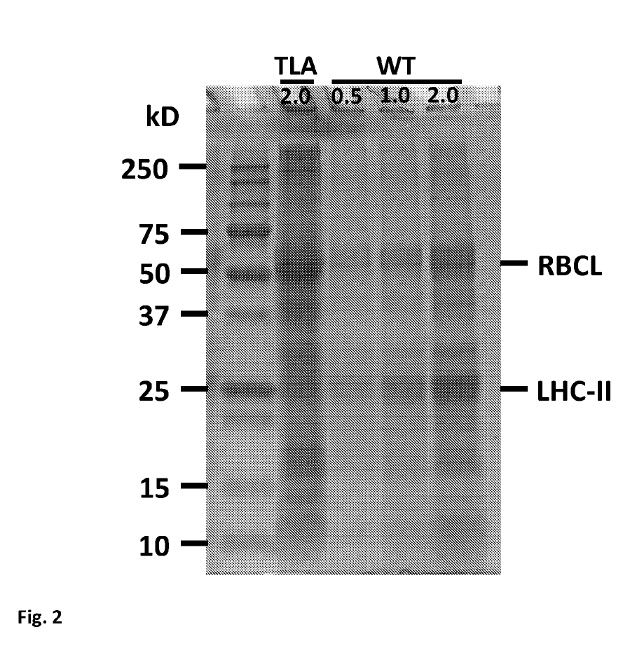

Isolated (intact) tobacco chloroplasts were solubilized and subjected to SDS-PAGE and Western blot analysis with specific polyclonal antibodies, cross-reacting with the PsaA-PsaB PSI-RC, the D2 (PsbD) PSII-RC protein, the LHCB1 light-harvesting protein, or the LHCB2 protein. Loaded on a Chl basis (0.5 to 2 μg), the SDS-PAGE Coomassie stain showed substantially lower levels of the LHC-II proteins in the TLA mutant than in the wild type (FIG. 2). At the same time, there was more Rubisco (RBCL) in the 1LA mutant than in the wild type.

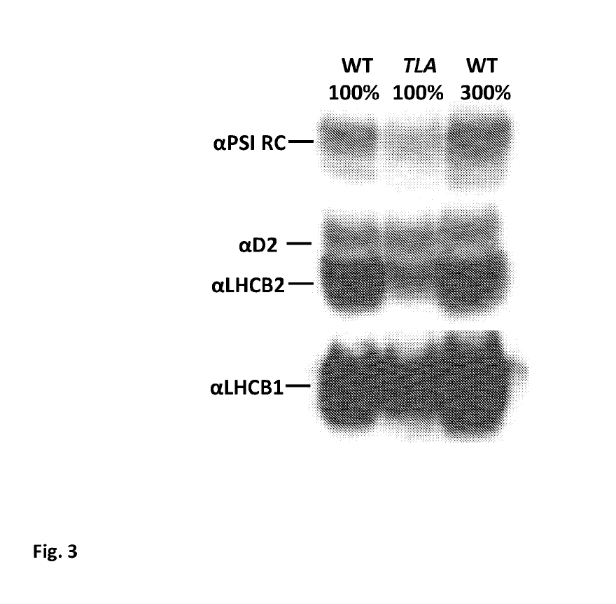

Western blot analysis showed that the TLA chloroplasts contain lower levels of the PsaA-PsaB PSI-RC proteins (FIG. 3), relative to the wild type, consistent with the greater PSII/PSI ratio measured spectrophotometrically (Table 2). Levels of the D2 (PsbD) PSII-RC protein were about the same, whereas levels of the LHCB1 and LHCB2 proteins were measurably lower in the TLA than wild type. These protein measurements are consistent with the smaller (truncated) light-harvesting Chl antenna size of the TLA mutant and corroborate the results of the spectrophotometric and kinetic analysis.

### Light-Saturation Curves of Photosynthesis

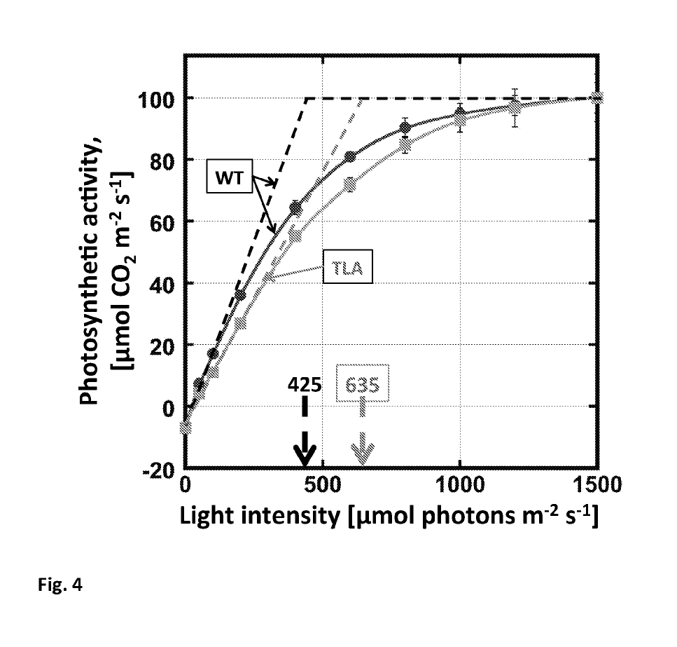

To investigate the functional properties of the photosynthetic apparatus in TLA tobacco relative to that in wild type, the light-saturation curve of photosynthesis was measured under in vivo conditions (FIG. 4). Results in FIG. 4 were normalized to the same light-saturated rate value to better illustrate differences in half-saturation intensity between the two samples. On a leaf surface area basis, the absolute dark respiration rate of wild type and TLA tobacco was about −2 μmol CO₂ m⁻² s⁻¹, whereas the absolute light-saturated rate Pₘₐₓ varied in different leaves between 20 and 35 μmol CO₂ m⁻² s⁻¹, depending on leaf development stage and probably other physiological conditions. On occasion, Pₘₐₓ of the TLA tobacco exceeded that of the wild type, when measured under ambient (400 ppm) CO₂ conditions. The reasons for this variation were not investigated further in the experiment.

Interestingly, in all cases examined, and irrespective of the Pₘₐₓ value attained, the photosynthesis-saturation intensity, measured from the intercept of the initially linear increase of the light-saturation curve with the Pₘₐₓ asymptotic line (dashed lines in FIG. 4) indicated a saturation intensity of 425 μmol photons m⁻² s⁻¹ for the wild type and 635 mol photons m⁻² s⁻¹ for the TLA mutant. This difference suggested a 1.5-fold difference in the light-harvesting antenna size of the photosystems in the two strains. Measured in this way, the apparent difference in the light-harvesting antenna size of the photosystems between the two strains is not as pronounced as that measured from the spectrophotometric and kinetic analysis (Table 2). Part of the discrepancy could be attributed to the specifics of the two measurements. In the spectrophotometric and kinetic analysis, only Chl a and Chl b molecules are sensitized by the weak green actinic illumination, whereas in the light-saturation curves of photosynthesis red and blue actinic light sensitizes both Chl and Car molecules in the light-harvesting antenna. TLA chloroplasts have a greater Car/Chl ratio than the wild type (Table 1), and Car excitation in the blue region of the spectrum may attenuate the difference in light harvesting between the two samples. In addition, increased penetration of light into the leaves of the TLA mutant might result in higher rates of photosynthesis because of improved light distribution within the leaf.

### Biomass Accumulation Under High Foliage-Density Conditions

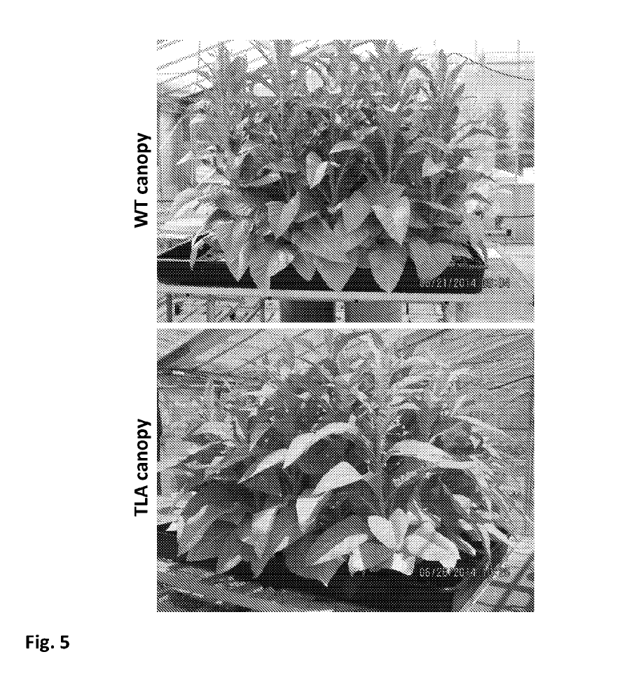

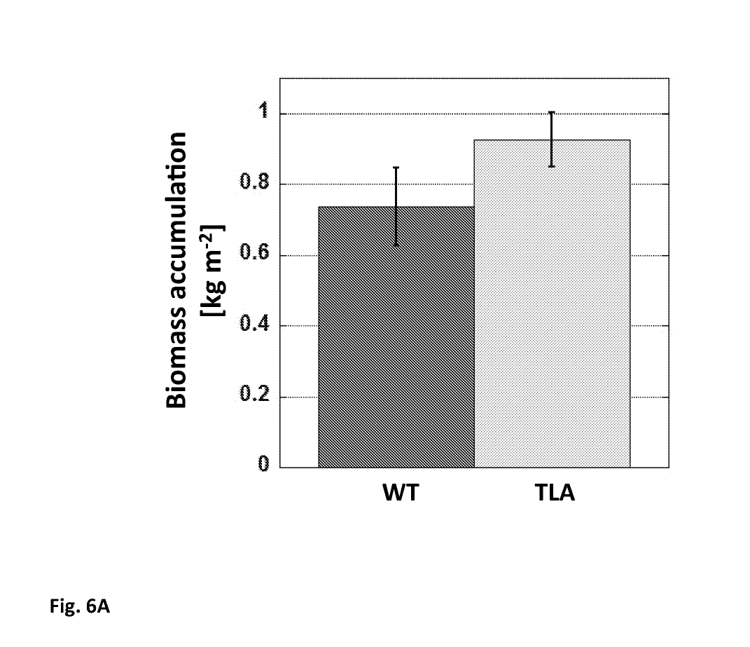

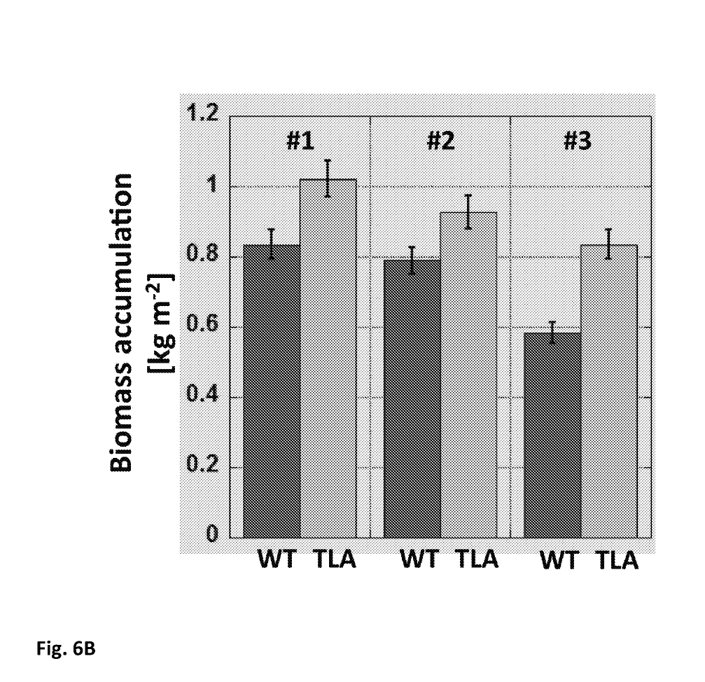

Three different monoculture sets comparing the growth and biomass accumulation of wild-type and. TLA tobacco plants were investigated in canopy density experiments. Comparative monoculture biomass accumulation measurements were conducted in the greenhouse during the natural tobacco growth season spanning the period from May to October. The layout of the monoculture entailed individual tobacco plants growing in 2-gallon pots, having a rim diameter of 9-inches. Pots were placed against one-another in a 5×5 monoculture configuration, comprising 25 plants separated by a 9-inch distance from each other. Relative to the wild type, TLA monocultures developed with a lag of 1-2 weeks, attributed to the slower development of seedlings in the greenhouse nursery. There were distinct visual differences in mature monoculture appearance between the wild type and tobacco. Wild-type plants in the monoculture tended to develop longer internode distances with lower density of leaves in their upper canopy than the TLA plants (FIG. 5). There was an overall higher foliage density in the TLA canopy, compared to the wild type. Importantly, there were differences in the total biomass that was harvested from the two types of monocultures. Wild-type biomass averaged 736±67 g dw m⁻² of monoculture surface area, whereas TLA biomass yield averaged 928±47 g dw m⁻² (FIG. 6A). There was variation among the absolute yields of the three separate pairs (FIG. 6B), depending on time of the year and greenhouse location, but in all cases the TLA monoculture produced more biomass than the corresponding wild type. In a one-tailed, paired statistical analysis of the biomass accumulation data, the calculated p-value was p=0.014, i.e., substantially lower than p=0.05, considered to be the threshold of statistically significant results. Thus, there was a notable 25% improvement in biomass accumulation, attributed to the better sunlight distribution through the TLA monoculture foliage, as compared to that of the wild type.

### TLA-Supported Agronomic Improvements

Application of the TLA concept to agriculture affords high density planting, thereby minimizing the surface area that is needed for the generation of a given amount of biomass. For example, traditional tobacco cultivation entails planting in rows with individual plants separated from each other by about 18 inches (FIG. 7). This practice translates into a density of 4.35 plants cultivated per m². Application of the TLA concept to tobacco, as applied in this work, would permit cultivation in rows with individual plants separated from each other by about 9 inches (FIG. 8). This improvement would increase monoculture density to 14.9 plants per m² with obvious benefits to yield per hectare.

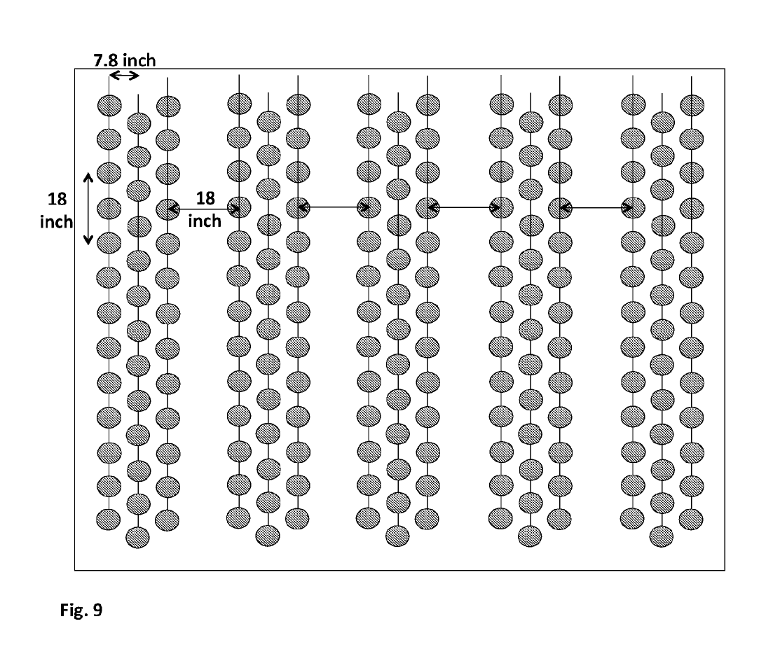

Practical considerations often dictate spacing for access to the plants in the field. A schematic of alternative tobacco agronomy with rows of three plants with an 18-inch distance between the groups of three is shown in FIG. 9. A relatively narrow distance of 9-inch still separates plants from each other within the group of three. This configuration with the 18-inch gap between rows of three translates into 12.7 plants per m². This planting density also benefits from the Truncated Light-harvesting Antenna (TLA) size of the photosystems, and of the ensuing lower chlorophyll content per leaf area, properties that afford a greater transmittance of sunlight through the foliage, thereby enhancing monoculture productivity.

Alternative planting configurations are also possible with two, four, or five rows of plants grouped together, with groups of rows separated by a space of 15, 18, or 23 inches to enable access to the plants throughout the field.

These agronomic improvements were exemplified in tobacco. However, the TLA principle can apply to other agricultural plants, as the TLA property is established in barley, soybean, corn, sugar beet, and possibly other crop species (Table 3). For example, favorable corn planting densities for high yield are in the range of 65,000 to 75,000 plants per ha (˜7 plants per m²). On the basis of the considerations described in this invention, this planting density could double to 140,000 plants per ha (14 plants per m²), substantially increasing crop yield. Similarly, currently optimal sugar beet planting density is about ˜100,000 plants per ha, (˜10 plants per m²), a density that could double to 200,000 plants per ha (20 plants per m²) upon application of the TLA-invention to this crop.

### Discussion of Examples

A challenge for plant scientists on a global scale is to generate enough food and feed for human and farm-animal nutrition, so as to meet the needs of an expanding world population (Godfray et al. 2010; Alexandratos and Bruinsma 2012). Several analyses of the photosynthetic solar-to-biomass energy conversion efficiency identified truncation of the light-harvesting antenna in photosynthesis as a potentially high dividend strategy for increasing crop productivity (Melis et al. 1999; Nagajima and Ueda 1999; Melis 2009; Ort et al. 2011, 2015; Kirst and Melis 2014). While assembly of large arrays of light-harvesting antenna pigments is a critical evolutionary adaptation and a survival strategy for plants competing to grow in limited light in the wild, this is strongly counterproductive in crop monocultures, where growth takes place under direct and excess sunlight. The large arrays of light harvesting antenna complexes in crop plants cause the upper canopies to over-absorb solar irradiance, far in excess of what is needed to saturate photosynthesis, forcing them to engage in wasteful dissipation mechanisms to deal with the excess energy. This property further causes shading of the inner and lower foliage leaves preventing them from attaining their maximum photosynthetic potential. The net effect of this large antenna configuration in crop plants is to lower the overall solar-to-biomass energy conversion efficiency of photosynthesis from a theoretically possible 8-10% to less than 1% (Melis 2009).

This example showed that the TLA technology could be applied to a crop monoculture, resulting in measurable improvement in biomass yield. The model plant Nicotiana tabacum (tobacco) was used in this example. However, the example is non-limiting, the TLA principle applies to other crop plants, promising to increase yields, while minimizing the space needed for cultivation. For example, soybeans are known to be subject to lower yields when planted at high densities (http://www.sugarresearch.com.au/icms_docs/157723_Bulletin_Vol_24_Sowing_soybean_seeds-get_the_rate_right.pdf). The lower yields observed with excessive sowing densities has been attributed to several factors, some of which could be mitigated upon application of TLA technology to this important crop. Declining yields with plant density are also seen in the case of corn, as documented by the University of Wisconsin, Agronomy Department (corn.agronomy.wisc.edu/AA/A062.aspx), a problem that could in part be alleviated with the application of TLA technology to this crop. Narrow rows of either soybean or corn offer additional advantages, as they achieve canopy closure more quickly, thus causing substantial shading of weed seedlings in the lower canopy. In addition, a high-density foliage can significantly reduce losses of soil moisture.

All publications, accession numbers, and patent applications cited in this specification are herein incorporated by reference as if each individual publication or patent application were specifically and individually indicated to be incorporated by reference.

### References Cited in the Disclosure by Author and Year

Abadia J, Glick R E, Taylor S E, Terry N and Melis A (1985) Photochemical apparatus organization in the chloroplasts of two Beta vulgaris genotypes. Plant Physiol. 79: 872-878

Alexandratos N, Bruinsma (2012) World Agriculture: Towards 2030/2050. The 2012 revision. ESA Working Paper No. 12-03 (Food Agric Org, Rome)

Anderson J M (1986) Photoregulation of the composition, function, and structure of thylakoid membranes. Annu Rev Plant Physiol 37:93-136

Arnon D I (1949) Copper enzymes in isolated chloroplasts: Polyphenol oxidase in Beta vulgaris. Plant Physiol 24:1-15

Fitzmaurice W P, Nguyen L V, Wernsman E A, Thompson W F, Conkling M A (1999) Transposon tagging of the sulfur gene of tobacco using engineered maize ac/ds elements. Genetics 153: 1919-1928

Ghirardi M L, McCauley S W and Melis A (986) Photochemical apparatus organization in the thylakoid membrane of Hordeum vulgare wild type and chlorophyll b-less chlorina f2 mutant. Biochim. Biophys. Acta 851:331-339

Ghirardi M L and Melis A (1988) Chlorophyll b-deficiency in soybean mutants. I.

Effects on photosystem stoichiometry and chlorophyll antenna size. Biochim. Biophys. Acta 932: 130-137

Godfray H C J, Beddington J R, Crute I R, Haddad L, Lawrence D, Muir J F, Pretty J, Robinson S, Thomas S M, Toulmin C (2010) Food security: the challenge of feeding 9 billion people. Science 32.7:812--818

Greene B A, Staehelin L A, Melis A (1988) Compensatory alterations in the photochemical apparatus of a photoregulatory, chlorophyll b-deficient mutant of maize. Plant Physiol 87: 365-370

Hansson A, Gamini Kannangara C, Von Wettstein D, Hansson M (1999) Molecular basis for semidominance of missense mutations in the XANTHA-H (42-kDa) subunit of magnesium chelatase. Proc. Natl. Acad. Sci. USA 96:1744-1749

Kirst H, Garcia-Cerdan J G, Zurbriggen A, Ruehle T, Melis A (2012) Truncated photosystem chlorophyll antenna size in the green microalga Chlamydomonas reinhardtii upon deletion of the TLA3-CpSRP43 gene. Plant Physiol. 160(4):2251-2260

Kirst H, Melis A (2014) The chloroplast Signal Recognition Particle pathway (CpSRP) as a tool to minimize chlorophyll antenna size and maximize photosynthetic productivity. Biotech Advances 32: 66-72

Kirst H, Formighieri C, Melis A (2014) Maximizing photosynthetic efficiency and culture productivity in cyanobacteria upon minimizing the phycobilisome light-harvesting antenna size. Biochim Biophys Acta—Bioenergetics 1837:1653-1664

Homann P H, Schmid G H (1967) Photosynthetic reactions of chloroplasts with unusual structures. Plant Physiol 42:1619-1632

Lichtenthaler H K (1987) Chlorophylls and carotenoids: pigments of photosynthetic biomembranes. Methods Enzymol 148:350-382

Melis A, Homann P H (1976) Heterogeneity of the photochemical centers in system II of chloroplasts. Photochem Photobiol 23: 343-350

Melis A, Thielen A P G M (1980) The relative absorption cross-section of photosystem I and photosystem II in chloroplasts from three types of Nicotiana tabacum. Biochim Biophys Acta 589: 275-286

Melis A, Brown J S (1980) Stoichiometry of system T and system II reaction centers and of plastoquinone in different photosynthetic membranes. Proc Natl Acad Sci USA 77: 4712-4716

Melis A (1989) Spectroscopic methods in photosynthesis: photosystem stoichiometry and chlorophyll antenna size. Phil Trans R Soc Lond B 323: 397-409

Melis A, Neidhardt J, Benemann J R (1999) Dunaliella salina (Chlorophyta) with small chlorophyll antenna sizes exhibit higher photosynthetic productivities and photon use efficiencies than normally pigmented cells. J Appl Phycol 10: 515-525

Melis A (2009) Solar energy conversion efficiencies in photosynthesis: minimizing the chlorophyll antennae to maximize efficiency. Plant Science 177: 272-280

Müller P, Li X-P, Niyogi K K (2001) Non-photochemical quenching: a response to excess light energy. Plant Physiol 125: 1558-4566

Mussgnug J H, Thomas-Hall S, Rupprecht J, Foo A, Klassen V, McDowall A, Schenk P M, Kruse O, Hankamer B (2007) Engineering photosynthetic light capture: impacts on improved solar energy to biomass conversion. Plant Biotech J 5:802-814

Nakajima Y, Ueda R (1997) Improvement of photosynthesis in dense microalgal suspension by reduction of light harvesting pigments. J Appl Phycol 9: 503-510

Nakajima Y, Ueda R (1999) Improvement of microalgal photosynthetic productivity by reducing the content of light harvesting pigments, J Appl Phycol 11: 195-201

Nakajima Y, Itayama T (2003) Analysis of photosynthetic productivity of microalgal mass cultures. J Appl Phycol 15:497-505.

Okabe K, Schmid G H, Straub J (1977) Genetic characterization and high efficiency photosynthesis of an aurea mutant of tobacco. Plant Physiol 60:150-156.

Ort D R, Zhu X G, Melis A (2011) Optimizing antenna size to maximize photosynthetic efficiency. Plant Physiol 155:79-85

Ort D R, Merchant S S, Alric J, Barkan A, Blankenship R E, Bock R, Croce R, Hanson M R, Hibberd J M, Long S P, Moore T A, Moroney J, Niyogi K K, Parry M A, Peralta-Yahya P P,

Prince R C, Redding K E, Spalding M H, van Wijk K J, Vermaas W F, von Caemmerer S, Weber A P, Yeates T O, Yuan J S, Zhu X G (2015) Redesigning photosynthesis to sustainably meet global food and bioenergy demand. Proc Natl Acad Sci USA 112:8529-8536.

Pone J E W, Benemann J R, Tanaka A and Melis A (2000) Photosynthetic apparatus organization and function in wild type and a Chl b-less mutant of Chlamydomonas reinhardtii. Dependence on carbon source. Planta 211: 335-344

Polle J E, Kanakagiri S D, Melis A (2003) tla1, a DNA insertional transformant of the green alga Chlamydomonas reinhardtii with a truncated light-harvesting chlorophyll antenna size. Planta 217:49-59

Ruban A V (2016) Non-photochemical chlorophyll fluorescence quenching: mechanism and effectiveness in protection against photodamage. Plant Physiol 170:1903-1916

Thielen A P G M, van Gorkom H L (1981) Quantum efficiency and antenna size of photosystem II-alpha, II-beta and I in tobacco chloroplasts. Biochim Biophys Acta 635:111-120

TABLE 1
Chlorophyll and carotenoid content per leaf area and pigment ratios of
Nicotiana tabacum wild type and TLA plants (n = 3-5; means ± SD)

| | WT | TLA | % change |
|---|---|---|---|
| Total Chl, mg cm⁻² of leaf area | 24.9 ± 2.1 | 12.1 ± 1.3 | 49 ± 6 |
| Chl a, mg cm⁻² of leaf area | 18.8 ± 1.6 | 10.7 ± 1.0 | 57 ± 7 |
| Chl b, mg cm⁻² of leaf area | 6.1 ± 0.6 | 1.4 ± 0.4 | 23 ± 11 |
| Chl a/Chl b ratio, mol:mol | 3.1 ± 0.1 | 8.1 ± 1.7 | — |
| Car, mg cm⁻² of leaf area | 4.1 ± 0.4 | 3.5 ± 0.6 | 85 ± 21 |
| Car/Chl ratio (w:w) | 0.16 ± 0.02 | 0.29 ± 0.05 | — |

TABLE 2
Photochemical apparatus characteristics of Nicotiana tabacum wild type
(WT) and TLA plants grown under ambient sunlight in high foliage density
conditions. Photosystem Chl antenna size and reaction center concentrations
were measured spectrophotometrically (Melis 1998). (n = 3; means ± SD.)

| | WT | TLA |
|---|---|---|
| Chl/Q_{A} (mol:mol) | 383 ± 8 | 129 ± 5 |
| Chl/P700 (mol:mol) | 412 ± 13 | 378 ± 29 |
| PSII/PSI (mol:mol) | 1.08 ± 0.04 | 2.93 ± 0.25 |
| K_{II} α [s⁻¹] | 8.4 ± 0.1 | 3.4 ± 0.1 |
| K_{II} β [s⁻¹] | 5.1 ± 0.2 | 2.4 ± 0.1 |
| K_{I} [s⁻¹] | 7.2 ± 0.4 | 3.0 ± 0.2 |
| Proportion PSII-α [%] | 46 ± 2 | 23 ± 3 |
| Proportion PSII-β [%] | 54 ± 4 | 77 ± 9 |
| N_{II} α (Number of Chl molecules specifically in | 242 ± 2 | 119 ± 3 |
| PSII-α) | | |
| N_{II} β (Number of Chl molecules specifically in | 148 ± 4 | 86 ± 2 |
| PSII-β) | | |
| N_{II} average | 191 ± 3 | 93 ± 2 |
| N_{I} (Number of Chl specifically in PSI) | 207 ± 12 | 104 ± 8 |

TABLE 3
Chlorophyll composition and chlorophyll antenna size of the photosystems
in selected Truncated Light-harvesting Antenna crop plants. Photosystem
Chl antenna size and reaction center concentrations were measured
spectrophotometrically (Melis 1998). (n = 3; means ± SD.) All
corresponding wild type strains showed a Chl a/Chl b ratio of about
3.0 ± 0.5. They possessed an average PSII antenna size in the range of
215-230 chlorophyll (a and b) and a PSI antenna size in the range of
180-210 chlorophyll (a and b) molecules. Genes that are mutated that
result in a small or truncated antenna size (TLA) property in green plants
and algae are known in the art and include TLA1 (U.S. Pat. No.
7,745,696), TLA2 (U.S. patent application 20140295448), TLA3 (Kirst
et al. 2012), CAO (Chlorophyltide a oxygenase, Ghirardi et al. 1986;
Polle et al. 2000), Mg-chelatase subunit I, CHL-I (Hansson et at. 1999;
Fitzmaurice et al. 1999).

| | | Average PSII | PSI antenna | |
|---|---|---|---|---|
| | Chl a/Chl b | antenna size | size* | |
| | ratio | (Chl | (Chl | |
| Plant species | mol:mol) | molecules) | molecules) | Reference |
| Hordeum vulgare | Chl b-less | 50 | 150 | Ghirardi et |
| (barley) | | | | al. 1986 |
| chlorina f2 | | | | |
| Glycine max | 5.0 | 135 | 160 | Ghirardi |
| (soybean) | | | | and Melis |
| y₉y₉ | | | | 1988 |
| Glycine max | 5.5 | 112 | 170 | Ghirardi |
| (soybean) | | | | and Melis |
| Y₁₁y₁₁ | | | | 1988 |
| Zea mays | 5.6 | 140 | 150 | Greene et al. |
| (maize) | | | | 1988 |
| OY-YG | | | | |
| Beta vulgaris | 5.2 | 120 | 120 | Abadia et al. |
| (sugarbeet) | | | | 1985 |
| PBI line LMG | | | | |

1. A method of obtaining plants having increased biomass accumulation, the method comprising cultivating a plurality of Truncated Light-harvesting Antenna (TLA) plants comprising individual TLA plants, wherein each of the TLA plants comprised by the plurality has a photosystem chlorophyll antenna size that is about 75% or less than the photosystem chlorophyll antenna size of a wild-type counterpart plant, wherein the TLA plants are planted:
at a density such that plant density of the TLA plants in a monoculture is increased by at least 20%, at least 50%, at least 100%, at least 200%, or at least 300% over the density employed for the counterpart wild type plants.

2. The method of claim 1, wherein the plants are tobacco or grape vine plants.

3. The method of claim 2, wherein the TLA plants are planted in a configuration where plant density is at least 4 plants per m², at least 8 plants per m², at least 12 plants/m², or at least 16 plants/m².

4. The method of claim 2, wherein the plants are in a configuration in which:
the plants are separated from one another by about 18 inches or less;
the plants are separated from one another by about 15 inches or less;
the plants are separated from one another by about 12 inches or less; or the plants are separated from one another by about 9 inches or less.

5. The method of claim 1, wherein the chlorophyll antenna size is about 75%, about 70%, about 60%, about 50%, about 30%, or about 25%, or less, of the chlorophyll antenna size of the counterpart wild type plant and at least 20% of the chlorophyll antenna size of the counterpart wild type plant.

6. The method of claim 5, wherein the chlorophyll a to chlorophyll b ratio of the leaves in the TLA plants is 4±1, 6±1, 8±1, 12±1, or 20±1, or greater; or where the TLA plants lacks chlorophyll b.

7. The method of claim 1, wherein the TLA plants are a species of dicots with broad leaves.

8. The method of claim 1, where in the TLA plants are tobacco, soybean, bean, potato, tomato, sorghum, sugar beet, cotton, canola, alfalfa, or grape vine plants.

9. The method of claim 1, wherein the TLA plants are monocots with blade-like leaves.

10. The method of claim 1, wherein the TLA plants are corn, wheat, barley, miscanthus, switchgrass, or Napier grass.

11. The method of claim 1, wherein the plants are cultivated under bright sunlight.

12. A method of enhancing biomass productivity on a per cultivated surface area, the method comprising cultivating a TLA variant of a crop or wild type plant under conditions in which the planting density of the TLA plants is greater by at least 25%, at least 50%, at least 100%, at least 200%, or at least 300% over that employed for the corresponding wild type plant.
